(19) United States
(12) Plant Patent Application Publication
(10) Pub. No.: US 2002/0170102 P1
(43) Pub. Date: Nov. 14, 2002

(54) Chrysanthemum plant named 'Fiji Orange Improved'

(76) Inventor: Mark Roland Boeder, West Wittering (GB)

Correspondence Address: Mark K. Dickson, HOWREY SIMON ARNOLD & WHITE, LLP, 750 Bering Drive, Houston, TX, 77057-2198, US

(21) Appl. No.: 09/851,653

(22) Filed: May 8, 2001

(51) Int. Cl. A01H 5/00

(52) U.S. Cl. PLT/290

(57) ABSTRACT

A new variety of Chrysanthemum plant named ‘Fiji Orange Improved,’ characterized by its flat capitulum form; early flowering; decorative type inflorescences, strong and long peduncles; numerous ray florets per inflorescence; numerous inflorescences per plant when grown as a spray; and orange ray florets.

### BACKGROUND OF THE INVENTION

The present invention comprises a new and distinct cultivar of Chrysanthemum botanically known as Dendranthema grandiflora, and referred to by the cultivar name ‘Fiji Orange Improved.’‘Fiji Orange Improved’, identified as CGC 97012, originated from a spontaneous whole plant mutation, found by Mark Boeder in a controlled testing program at Chichester, West Sussex, United Kingdom of the variety ‘White Fiji’ (U.S. Plant Pat. No. 10,066). The new variety ‘Fiji Orange Improved’ has been asexually reproduced by vegetative cuttings at Chichester, West Sussex, United Kingdom and the distinguishing characteristics are retained through successive generations of asexual reproduction.

### BRIEF SUMMARY OF THE INVENTION

‘Fiji Orange Improved’ is a cut type of Chrysanthemum plant variety having a flat capitulum form, decorative type inflorescences and orange ray florets.

### Comparison with Parent

Plants of the new Chrysanthemum variety ‘Fiji Orange Improved’ are similar to the cultivar ‘White Fiji’ in plant habitat and growth rate. In side-by-side comparisons at Chichester, West Sussex, United Kingdom, under commercial practice, plants of the new Chrysanthemum variety ‘Fiji Orange Improved’ differed from plants of the parent cultivar ‘White Fiji’ in the following characteristics.

1. The new variety ‘Fiji Orange Improved’ produces orange flowers whereas the original cultivar ‘White Fiji’ produces white flowers.

### Comparison with Other Varieties

Plants of the new Chrysanthemum variety ‘Fiji Orange Improved’ are similar to the cultivar ‘Bright Yellow Fiji’ (U.S. Plant Pate. No. 10,236) in flower type, growing rate and reaction time. However, in side-by-side comparisons at Chichester, West Sussex, United Kingdom, under commercial practice, plants of the new Chrysanthemum variety ‘Fiji Orange Improved’ differed from plants of the cultivar ‘Bright Yellow Fiji’ in the following characteristics.

1. The new variety ‘Fiji Orange Improved’ produces orange flowers whereas the cultivar ‘Bright Yellow Fiji’ produces yellow flowers.

2. Plants of the new variety ‘Fiji Orange Improved’ have a longer inflorescence than plants of the cultivar ‘Bright Yellow Fiji’.

3. Plants of the new variety ‘Fiji Orange Improved’ have a response time which is two days faster than plants of the cultivar ‘Bright Yellow Fiji’.

### BRIEF DESCRIPTION OF ILLUSTRATIONS

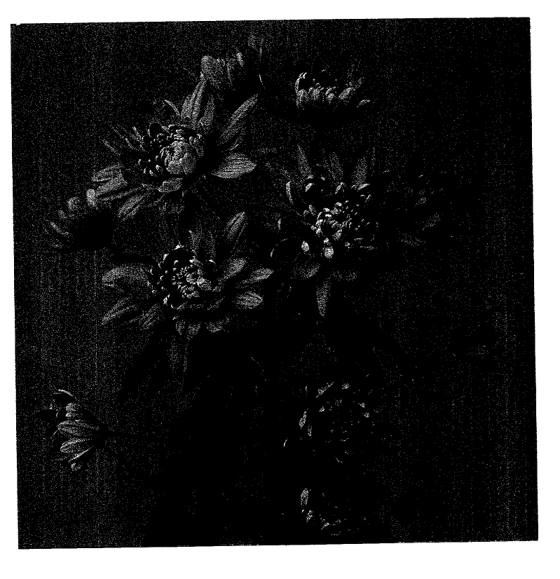

Typical specimens of the plant and flowers for the new Chrysanthemum variety ‘Fiji Orange Improved’ are shown in the accompanying photograph. The colors shown are as true as possible within the usual limits of this kind of illustration. FIG. 1 comprises a side perspective view of a typical flowering stem of the new variety ‘Fiji Orange Improved’ grown as a cut flower.

### DETAILED BOTANICAL DESCRIPTION

The following description of the new Chrysanthemum variety ‘Fiji Orange Improved’ is of plants grown in a glass covered greenhouse at Chichester, West Sussex, United Kingdom in the month of March. The cultivar has not been observed under all possible environmental conditions. The phenotype may vary significantly with variations in the environment such as temperature, length of day and light intensity, without any variance in genotype. The commercial classification of the new variety is a cut Chrysanthemum.

Plants of the new variety have been grown successfully under temperature conditions averaging about 18° C. at night and about 18° C. to 24° C. during the day under light conditions of about 3,000 (cloudy conditions) to 8,000 (sunny conditions) foot candles. The plants respond well to the use of growth retardant, such as one B9 treatment at about 2 gram/liter. To produce a commercial product the plants may be pinched once or may be grown without a pinch. It has been observed that the shelf life of the new variety is about 21 days with a response time of about 7.5 weeks.

The new variety may be produced as a spray (center bud removed) or disbud (lateral buds removed). The following description is with respect to a plant produced as a cut flower without pinching. In the description of this new Chrysanthemum variety, color values have been taken from The Royal Horticultural Society Colour Chart (R.H.S.C.C.).

Plant:

Plant type.—decorative type cut chrysanthemum.

Habit.—upright.

Height.—75-110 cm.

Response time.—7.5 weeks.

Vigor.—moderate.

Shelf life.—near 21 days.

Disease (susceptibility or resistance observed).—no observations.

Growth retardant type and treatment.—1 application B9 after 3 weeks in short days (13 hours of darkness) of 2 gram/liter.

Propagation.—Type: terminal tip cuttings Time to rooting—14 days with soil temperatures of 22° C. Rooting habit—uniform, fine, fibrous and well branched.

Foliage:

Compound or single.—single.

Arrangement of leaves.—alternate Shape of Leaf: typical 5 lobed Size of Leaf: Width (cm): 5.0-6.0 Length (cm): 8.0-9.5 Leaf Apex: Mucronate Base: Cordate Margin: palmately lobed Surface characteristics: Top: slightly pubescent Bottom: slightly pubescent

Petiole.—Color: 147A Length: 1.5-2.0 cm.

Venation (describe).—prominent on under surface Color: upper side: 147B underside: 147B.

Color.—Mature Leaf, upper side: 147A under side: 147B Young Leaf, upper side: 147A; under side: 147B.

Flower:

Flower type.—decorative.

Number of blossoms per branch.—8-14.

Inflorescence form.—cyme Depth of fully expanded blossoms: 1.2-2.0 cm Diameter of fully expanded blossoms: 1.3-2.0 cm Anemone center diameter: n/a.

Sepals.—Number: 15-25 Color: near 137A Length: 0.6-1.1 cm Width: 0.4-0.6 cm.

Peduncle.—Peduncle length is shortest at the terminal flower and progressively longer moving down the stem; longest length is on average 14-17 (cm), shortest length is on average 11-13 (cm) Length: 11-17 cm Color: 137B Surface: pubescent Texture: glabrous.

Ray florets.—Form/Shape: elongated Texture/Appearance: satiny, smooth Number per flower: 250-300 Length: 3.0-4.0 cm Width: 1.1-1.6 cm Apex: dentate Base: acute Margin: entire

Disc florets.—Form/Shape: tubular Number per flower: 5-15 Length: about 2 mm; Width: about 1 mm Diameter of disc: <0.5 cm.

Fragrance.—moderate.

Flower bud (at onset of color).—Length: 0.8-1.0 cm Diameter: 1.0-1.5 cm Form/shape: spherical.

General flower color.—1. Ray florets, upper side: Immature: near 34A Mature: centre near 34B, tips near 32C Older/Fading: near 34C 2. Ray florets, under side: Immature: near 20B Mature: near 20B Older/Fading: near 20C 3. Disc florets: Immature: 154A Mature: 12B.

Flower progression with age.—Color fades slightly when flowers age.

Reproductive Organs:

Ray florets.—Pistillate Style Color: 151B Style Length: 0.1-0.3 cm

Disc florets.—Pistillate Style Color: 151B Style Length: 0.1 cm Staminate: Anther Shape: thread Anther Color: 12A Pollen Quantity: moderate

What is claimed is:

1. A new and distinct variety of Chrysanthemum plant, substantially as described and illustrated herein.
